(19) United States
(12) Patent Application Publication
(10) Pub. No.: US 2026/0199264 A1
(43) Pub. Date: Jul. 16, 2026

(54) COMPOSITIONS AND METHODS FOR APPETITE SUPPRESSION, WEIGHT LOSS, AND CONTROL OF BLOOD SUGAR

(71) Applicant: Miami Pharma Patent Holdings, Sheridan, WY (US)

(72) Inventor: Matthew Shin, Miami, FL (US)

(21) Appl. No.: 19/024,808

(22) Filed: Jan. 16, 2025

(51) Int. Cl. A61K 31/137; A61K 31/155; A61K 31/202

(52) U.S. Cl. CPC A61K 31/137; A61K 31/155; A61K 31/202

(57) ABSTRACT

The disclosure is directed to pharmaceutical compositions including Phentermine in an amount ranging from about 10 mg to about 37.5 mg, Metformin in an amount ranging from about 250 mg to about 2500 mg, and Omega-3 in an amount ranging from about 1 g to about 5 g. The pharmaceutical composition can be in the form of a pill, tablet, or capsule. The disclosure further includes therapies and methods for appetite suppression, controlling weight loss, or controlling blood sugar including administering to a mammal in need thereof a pharmaceutical composition including Phentermine in an amount ranging from about 10 mg to about 37.5 mg, Metformin in an amount ranging from about 250 mg to about 2500 mg, and Omega-3 in an amount ranging from about 1 g to about 5 g. The mammal may be a human.

### BACKGROUND

### Technical Field of the Invention

The disclosure relates to pharmaceutical compositions and methods for appetite suppression, weight loss, and the control of blood sugar that include phentermine, metformin, and omega-3.

### Description of the Related Art

Pharmaceutical compositions for appetite suppression, weight loss, and control blood sugar are known, however, in currently available formulations and treatments the effectiveness of the compositions is not as high as it could be and there are often unacceptable side effects.

### SUMMARY DISCLOSURE OF THE INVENTION

The disclosure is directed to pharmaceutical compositions including Phentermine in an amount ranging from about 10 mg to about 37.5 mg, Metformin in an amount ranging from about 250 mg to about 2500 mg, and Omega-3 in an amount ranging from about 1 g to about 5 g. The pharmaceutical composition can be in the form of a pill, tablet, or capsule. The pharmaceutical composition can be in the form of a nano pill. The disclosure further includes therapies and methods for appetite suppression, controlling weight loss, or controlling blood sugar, comprising: administering to a mammal in need thereof a pharmaceutical composition including Phentermine in an amount ranging from about 10 mg to about 37.5 mg, Metformin in an amount ranging from about 250 mg to about 2500 mg, and Omega-3 in an amount ranging from about 1 g to about 5 g. The mammal may be a human.

Other features and aspects will be apparent from the following detailed description and the claims.

### DETAILED DISCLOSURE OF THE INVENTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

### Modes for Carrying Out the Invention

In embodiments, the present disclosure provides pharmaceutical compositions that include Phentermine, Metformin, and Omega-3 that suppress appetite and control blood sugar. In embodiments, the composition can be included in a pharmaceutical composition e.g., a capsule, pill, or tablet. In embodiments, the pill can be a nano pill.

The phentermine can range from 10 mg to 37.5 mg in the composition (e.g., per capsule, pill, or tablet); likewise the Metformin can range from 250 mg to 2500 mg; and likewise the omega-3 can range from 1 g to 5 g.

The disclosure also provides methods of administering the disclosed pharmaceutical compositions for appetite suppression, controlling weight loss, or controlling blood sugar. In one embodiment, a daily dosage range could be 10 mg to 37.5 mg of phentermine, 250 mg to 2500 mg of Metformin, and 1 g of omega-3. The phentermine, Metformin, and omega-3 therapy can be administered in a single composition (e.g., capsule, pill, or tablet) or in multiple compositions, e.g., capsules, pills, or tablets to provide the required daily dosage. A daily therapy, for example, could include one tablet, pill, capsule in the morning and one or two in the evening to provide a daily dosage within the above ranges.

In embodiments, the compositions can include or comprise Phentermine, Metformin, and Omega-3 in order to provide appetite suppression, control weight loss, and/or control blood sugar.

In embodiments, the compositions can consist essentially of Phentermine, Metformin, and Omega-3. In this case, the Phentermine, Metformin, and Omega-3 would be the active ingredients of the composition. It is understood that a composition consisting essentially of Phentermine, Metformin, and Omega-3 can include further components that are commonly included in pharmaceutical or dietary supplement compositions such as carriers, diluents, adjuvants, solubilizing agents, suspending agents, fillers, surfactants, antimicrobial agent(s), preservative(s), viscosity modifier(s), thixotropy modifier(s), wetting agent(s), emulsifier(s), and the like.

In embodiments, the compositions can consist of Phentermine, Metformin, and Omega-3. In this case, each component is included at purity levels that are commercially available. The consisting of transitional phrase would not exclude common impurities, side products, and the like, that could be present, for example, due to the manufacturing process.

The compositions can be in the form of a liquid, gel, suspension, dispersion, solid, emulsion, aerosol, powders, tablets, capsules, and pills. Also, the compositions disclosed herein can be in a form suitable for dilutions. Similarly, the compositions can be in the form of a cream or paste. In a preferred embodiment, the compositions are included in the form of a pill, tablet, or a capsule. The pills can be nano pills. These above pharmaceutical terms are used consistent with current usage in the pharmaceutical arts as evidenced, for example, by Remington's Pharmaceutical Sciences, Sixteenth Edition, E. W. Martin (Mack Publishing Co., Easton, Pa., 1980); and Remington: Essentials of Pharmaceutics, Pharmaceutical Press; 1st edition (Apr. 1, 2013).

The three components are not known for any interaction risks in pharmacy databases.

### Glossary

Phentermine (dimethyl-phenethylamine) is a composition that has been used to treat obesity.

Metformin (N, N-dimethyl-biguanide) is known for the treatment of type 2 diabetes. It is also used in the treatment of polycystic ovary syndrome and has been used to lessen the risk of metabolic syndrome in people who take antipsychotic medication. It is also known as an anti-inflammatory.

Omega-3 fatty acids (omega-3 oils, ω-3 fatty acids or n-3 fatty acids) are polyunsaturated fatty acids that have a double bond three atoms away from the terminal methyl group in their chemical structure. Omega-3 fatty acids have been used to reduce the risk of cancer or cardiovascular disease. Fish oil supplements containing omega-3 fatty acids have been used to reduce the risk of heart attacks, strokes, and vascular diseases.

A tablet or a pill is an oral dosage form that typically comprises a solid dosage with optional excipients. A tablet or a pill may also include liquids, syrups, elixirs, suspensions, and emulsions as well. See e.g., Tablet (pharmacy), Wikipedia, the free encyclopedia, of record, herein incorporated by reference.

A capsule refers to a dosage form that is used to enclose a medicine in a relatively stable shell. Capsules be taken orally or can be used as suppositories. Capsules include hard-shelled capsules, which contain dry, powdered ingredients or miniature pellets. These can be made in two-parts, i.e., one part that is filled and then sealed using another part. Soft-shelled capsules can be used for oils and for active ingredients that are dissolved or suspended in oil.

The term “about” as used herein generally refers to compositional amounts that can vary by plus or minus 20%.

The term “mammal” as used herein are a group of vertebrate animals constituting the class Mammalia. See Mammal, Wikipedia, the free encyclopedia, of record, herein incorporated by reference. Mammal preferably refers to primates and most preferably humans.

The term “administration” generally includes oral and intravenous administration as well as any route of administration capable of effectively delivering the composition to the body. Preferred would be pills, capsules, or tablets. Administration may also be done through a food or beverage or through the skin or other body cavity. Administration may be through a parenteral route. Parenteral administration can be performed by injection (typically a hypodermic needle) and a syringe or through an indwelling catheter. See Route of Administration, Wikipedia, the free encyclopedia, of record, herein incorporated by reference.

The term “therapy” refers to the attempted remediation of a condition or health problem. Treatment or therapy can include providing relief to, preventing, curing, supporting, or maintaining a certain state with respect to a condition or management of a condition. Conditions can include weight loss therapy, appetite suppression, and controlling blood sugar, e.g., treating type 2 diabetes. Therapy can include prevention, management, e.g., halting or slowing the condition's development and effects, and relieving the symptoms of, as well as curing or eradicating the condition. See e.g., Therapy, Wikipedia, the free encyclopedia, of record, herein incorporated by reference.

An active ingredient or active substance in a composition is an ingredient or substance that is biologically active. In embodiments, the compositions of the disclosure have more than one active ingredient. Excipients are generally biologically inactive ingredients, although need not necessarily be inert. See e.g., Active Ingredient, Wikipedia, the free encyclopedia, of record, herein incorporated by reference.

A nano composition is a pharmaceutical composition utilizing the medical application of nanotechnology. Nanomedicine includes the medical applications of nanomaterials and biological devices. Functionalities can be added to nanomaterials by interfacing them with biological molecules or structures. Nanomedicines and materials include nano drug delivery vehicles.

While this disclosure includes specific examples, it will be apparent after an understanding of the disclosure of this application has been attained that various changes in form and details may be made in these examples without departing from the spirit and scope of the claims and their equivalents.

1. A pharmaceutical composition comprising:
Phentermine in an amount ranging from about 10 mg to about 37.5 mg;
Metformin in an amount ranging from about 250 mg to about 2500 mg;
omega-3 in an amount ranging from about 1 g to about 5 g.

2. The pharmaceutical composition of claim 1, wherein the pharmaceutical composition is in the form of a pill, tablet, or capsule.

3. A method for appetite suppression, controlling weight loss, or controlling blood sugar, comprising: administering to a mammal in need thereof a pharmaceutical composition of claim 1.

4. The method of claim 3, wherein the mammal is a human.

5. The method of claim 3, wherein the daily dosage of phentermine is about 10 mg to about 37.5 mg; the daily dosage of metformin is about 250 mg to about 2500 mg; and the daily dosage of omega- 3 is about 1 g to about 5 g.

6. The method of claim 3, wherein the pharmaceutical composition is in the form of a pill, tablet, or capsule.

7. The method of claim 6, wherein the pharmaceutical composition is in the form of a pill.

8. The method of claim 6 wherein the administration is a parenteral administration.

9. The method of claim 8, wherein the parenteral administration is an injection.
